(19) United States
(12) Patent Application Publication
(10) Pub. No.: US 2013/0006374 A1
(43) Pub. Date: Jan. 3, 2013

(54) KNEE PROSTHESIS HAVING A MIXED MENISCAL PLATE

(75) Inventors: Regis Le Couedic, Bordeaux (FR); Denis Pasquet, Quinsac (FR)

(73) Assignee: Implanet, Martillac (FR)

(21) Appl. No.: 13/583,701

(22) PCT Filed: Mar. 16, 2011
(86) PCT No.: PCT/FR2011/000148

§ 371 Date: Sep. 10, 2012

(30) Foreign Application Priority Data
Mar. 16, 2010 (FR) 10/01056

(51) Int. Cl. A61F 2/38

(52) U.S. Cl. 623/20.28

(57) ABSTRACT

The invention relates to a knee prosthesis (1) including a femoral part (2) connected to a tibial plate (3) by an intermediate meniscal plate (4) provided with an upper surface (5). The intermediate meniscal plate (4) and the tibial plate (3) are connected to each other by two planar joining surfaces, the tibial plate (3) including a central pivot pin (26) or at least one clipping/indexing finger (34, 35) which protrudes with respect to the tibial joining surface (11). The meniscal joining surface (12) of the meniscal plate (4) includes, in combination, a central hole (25) capable of engaging with the pivot pin (26) of the tibial plate in one case, and a locking recess (46, 47) capable of becoming embedded with the indexing finger (34, 35) of the tibial plate in the other one case, depending on whether the tibial plate comprises a pivot pin or an indexing finger.

The present invention relates to a knee prosthesis comprising a femoral component, a tibial plateau and an intermediate meniscal plate.

It has an important application mainly, but not exclusively, in the field of so-called total knee prostheses.

It is known that knee prostheses are generally of the three-compartment type.

In other words, they are prostheses comprising, on the one hand, two elements for replacing the femoral part and tibial part of the joint between the femur and the tibia, and, on the other hand, an element for forming the intermediate friction surface of the patella.

The complete or total prosthetic structure thus has a femoral component or plane, a tibial plateau or plane, and a meniscal plate often called the tibial insert. It is completed by a patellar implant which, since it has no role in the context of the present invention, will not be mentioned again hereinafter.

In a manner known per se, the tibial plateau cooperates with the femoral component via the meniscal plate, so as to thereby produce the different movements of the knee joint.

It should he noted at this point that, in order to permit adaptation to the different morphologies of patients, all of these implants have to be provided in several sizes and thicknesses. Regarding the use of these prostheses, there are essentially two techniques for reconstructing the knee joint.

The first technique involves using a meniscal plate that is fixed with respect to she tibial plateau.

The second technique involves using what are called mobile meniscal plates, as opposed to the fixed meniscal plates.

These mobile plates are designed to better reproduce the Kinematics of the joint by reducing the paradoxical movements due to the femoral component.

To permit the implementation of these two techniques, the surgeon must therefore have access to meniscal plates for each size and for each possible technique, and also in several thicknesses.

Moreover, regardless of the meniscal plates used, whether fixed or mobile, some surgeons consider it important to preserve the posterior cruciate ligament, when possible, in order to limit the anterior displacements of the femur with respect to the tibia.

By contrast, other surgeons favor removing this ligament, preferring to achieve limitation of the anterior displacement of the femur on the basis of the particular design of the meniscal plate.

It is therefore necessary to have fixed plates with cruciate, fixed plates without cruciate, mobile plates with cruciate and mobile plates without cruciate, which further increases the number of plates. It will be noted that “without cruciate” is equivalent to “postero-stabilised”.

The surgeon then has to choose the meniscal plate that will permit optimal reconstruction of the patient's joint in terms of the stability and amplitude of the movements wanted for the patient in question.

The prostheses of the prior art have a considerable disadvantage in particular in this respect. They require the provision of a very large number of femoral components, tibial plateaus and meniscal plates, in order to cover all eventualities.

In other words, a range of knee implants which includes the two types of meniscal plates, namely the mobile plates and the fixed plates, while permitting a choice between preserving the posterior ligament or removing it, is necessarily composed of a large number of components.

For example, a range of prostheses in the prior art consisting of eight sizes of femoral components for each side and for each type, i.e. thirty-two femoral components in total, three thicknesses of meniscal plates for each size and each type, and, finally, sixteen sizes of tibial plates, requires the provision of one hundred and forty-four different elements.

Prostheses are known (US 2006/0161259) that comprise a central fixation component and are intended to function after removal of the ligament.

Prostheses are also known (EP 0 732 091) that are designed to function without removal and with antero-posterior play and the possibility of rotation.

Such prostheses give rise to paradoxical movements leading to dislocations of the joint and to abnormal attrition of the meniscal component.

Moreover, none of these prostheses allows control of both clinical cases simultaneously.

One of the objects of the present invention is to make available a knee prosthesis which meets the practical requirements better than those that are already known, especially one which permits the same physical possibilities as the prostheses of the prior art, but which requires a much smaller number of elements.

Thus, practically all of the component parts of a prosthesis according to the invention will be able to function in one case with removal of the ligament and in the other case without removal of the ligament.

By virtue of one of the aspects of she invention, it will thus be possible to reduce the stock and therefore the investments of the implant manufacturer, while at the same time increasing patient safety by eliminating potential sources of error.

Moreover, the invention reduces the operating time and allows surgeons and operating theater personnel to be trained quickly in the use of the range of implants.

To this end, the invention essentially proposes a knee prosthesis comprising a femoral component connected to a tibial plateau by an intermediate meniscal plate provided with an upper face, said femoral component having a trochlear shield provided with an outer face cooperating under gentle friction with at least one guide area of complementary shape formed in said upper face,
characterized in that the intermediate meniscal plate and the tibial plateau are connected to each other by way of two planar joining faces, namely a tibial joining face and a meniscal joining face, the tibial joining face being larger than the meniscal joining face in its antero-posterior dimension and medio-lateral dimension,
in that the tibial plateau comprises a central pivot pin or at least one clipping/indexing finger, said pin or said finger protruding with respect to the tibial joining face,
and in that the meniscal joining face of the meniscal plate comprises, in combination, a central blind hole capable of cooperating under gentle friction about its entire periphery with the pivot pin of the tibial plateau in one case, and a locking recess capable of being engaged by the indexing finger of the tibial plateau in the other case, depending on whether the tibial plateau has a pivot pin or an indexing finger.

Thus, by virtue of this particular design of the meniscal plate, the same meniscal plate, which can also be designated as a so-called mixed meniscal plate, can be used equally as a fixed plate or as a mobile plate, depending on the tibial plateau chosen by the surgeon, and this will reduce the stock of meniscal plates by half.

In the embodiment more particularly described here, the tibial joining face is larger than the meniscal joining face in all of its dimensions parallel to its antero-posterior axis of symmetry, and also perpendicularly with respect to this axis (medio-lateral dimensions).

In other words, when the meniscal plate is centered, and in its median position on the tibial plateau, all of the meniscal joining surface is inscribed entirely within the tibial joining surface, which has an edge always protruding, for example by 1 mm, beyond the periphery of the meniscal joining surface, this periphery never being coincident at any point with said edge in this position.

This arrangement will, on the one hand, surprisingly permit the standardization of the components leading to the invention and will, on the other hand, permit protection of the ligaments and/or soft tissue parts belonging to and/or adjacent to the joint.

Advantageously, the surfaces of the planar joining faces are in the shape of an ellipse or substantially in the shape of an ellipse, truncated on an edge, for example over 1/10th or even 1/20th of their surface, parallel to the main axis of the ellipse.

The surfaces are designed to permit rotary pivoting of one with respect to the other, such that the curved outer periphery of the meniscal joining face in the shape of a portion of an ellipse, or substantially in the shape of a portion of an ellipse, remains inscribed within the joining face of the tibial plate, for an angle of rotation between the faces of between −12° and +12°, or even −8° and +8° and/or −4° and +4°, with respect to the medio-lateral axis of the meniscal plate, or main axis of the ellipse.

In practice, with the movements of the knee being physiologically limited in terms of rotation of the order of ±4 to 5°, or ±7 to 8°, such arrangements make it possible to preserve the soft tissue parts without the need to provide specific limit stops.

Unexpectedly, therefore, there is natural protection of the soft tissue parts.

Moreover, the fact that the (potentially aggressive) outer periphery of the meniscal plate, thus always inscribed within the surface of the tibial plate, is able to pivot in rotation about a pin joined integrally to the tibial plateau or tibial component, without any possibility of the meniscal plate sliding with respect to the tibial plateau, or by contrast can be rigidly fixed to said tibial component by fingers, avoids the protuberances that occur in the position of equilibrium and that create micro-stresses leading to more rapid wear and/or to pain.

In particular embodiments, use is also made of one and/or more of the following arrangements:
the upper face of the meniscal plate is provided with a pin and with two guide areas formed in the upper face on each side of said pin, the trochlear shield of the femoral face having a slit for guiding said pin;
the tibial plateau comprises at least one indexing finger, designed to engage in the recess for locking the meniscal plate in a defined position with respect to the tibial plateau;
the tibial plateau comprises two posterior indexing fingers and one anterior indexing finger able to cooperate with two posterior recesses and one anterior recess of the meniscal plate;
the tibial joining face has a peripheral shoulder forming a clipping finger along at least part of the periphery of said plateau in which the meniscal joining face engages completely;
the tibial plateau comprises a central pivot pin, the meniscal plate being mounted so as to be movable in rotation with respect to the tibial plateau about said pin;
the pin of the upper face of the meniscal plate has the shape of a Phrygian cap or a thumb;
the Phrygian cap has an upper lip forming a slight projection designed to generate a retreating movement of the femoral component in the event of its shifting by more than 1 mm with respect to the meniscal plate.

Such an arrangement avoids anterior dislocation of the prosthetic joint;
the meniscal joining face has at least one recess angled with respect to the periphery of said face and designed to permit the disconnection of the meniscal plate from the tibial plateau. This recess, which can be accessed laterally by the surgeon with an instrument, allows him to disconnect them or unclip them from each other by leverage;
the zone posterior to the central pin of the meniscal plate has a reinforcing part connecting the two postero-lateral zones of articular friction;
the femoral component and the tibial plateau are made of metal alloy, and the meniscal plate is made of polymer plastic, for example polyethylene.

The invention also proposes a set of several prostheses as have been described above, characterized in that it has at least one tibial plateau for a fixed meniscal plate and at least one tibial plateau for a mobile meniscal plate, and a range of corresponding meniscal plates of several thicknesses.

The invention will be better understood on reading the following description of embodiments given as non-limiting examples.

In the description, reference is made to the accompanying FIGS. where:

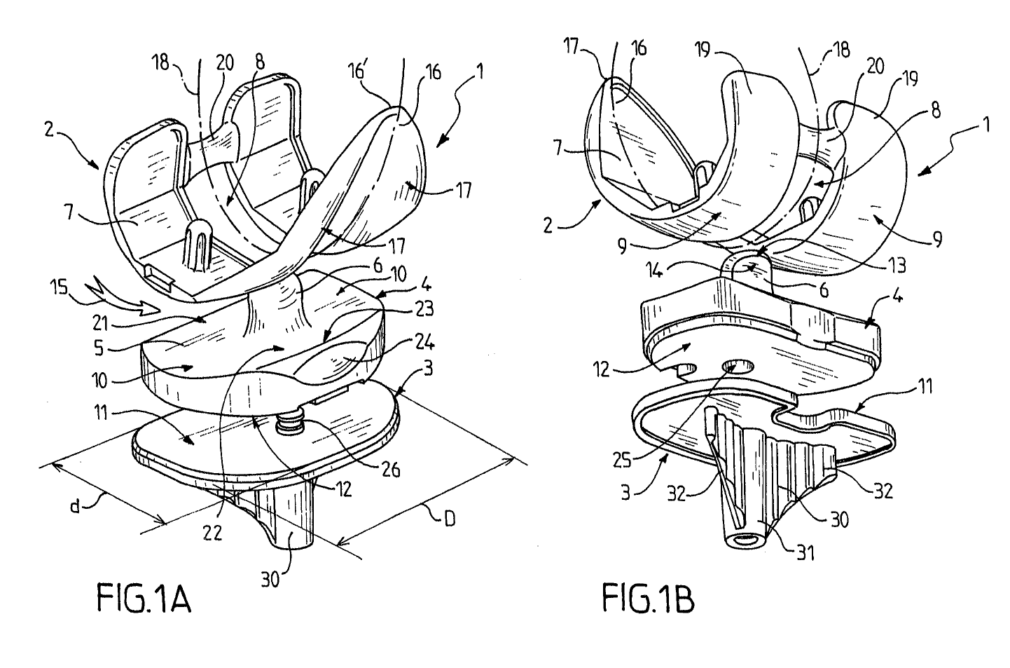

FIGS. 1A and 1B show perspective views, respectively from above and from below, of the three elements, detached from one another, of a prosthesis according to a first embodiment of the invention.

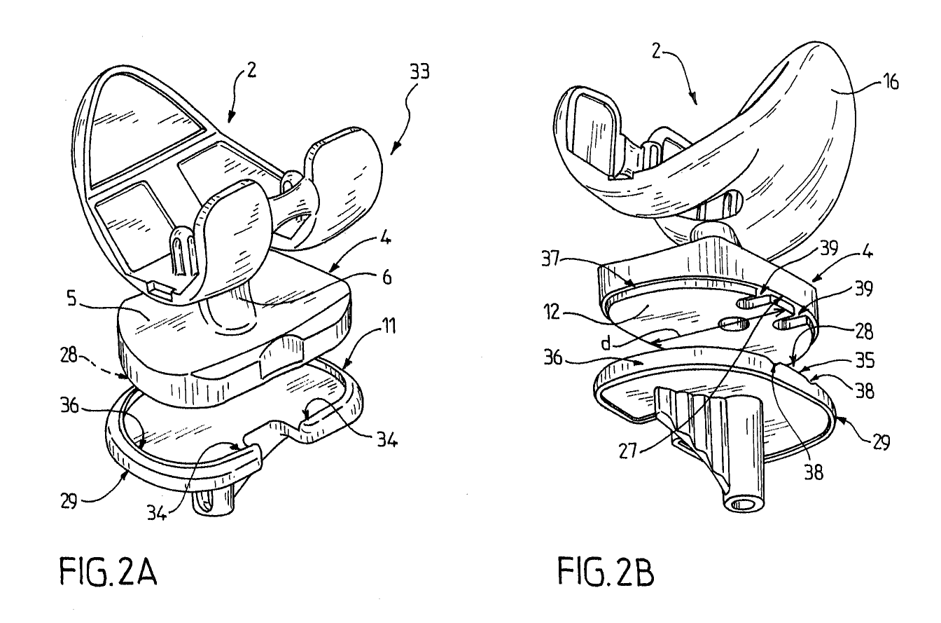

FIGS. 2A and 2B show perspective views, respectively from above and from below, of a second embodiment of a prosthesis according to the invention, with the elements once again detached from one another.

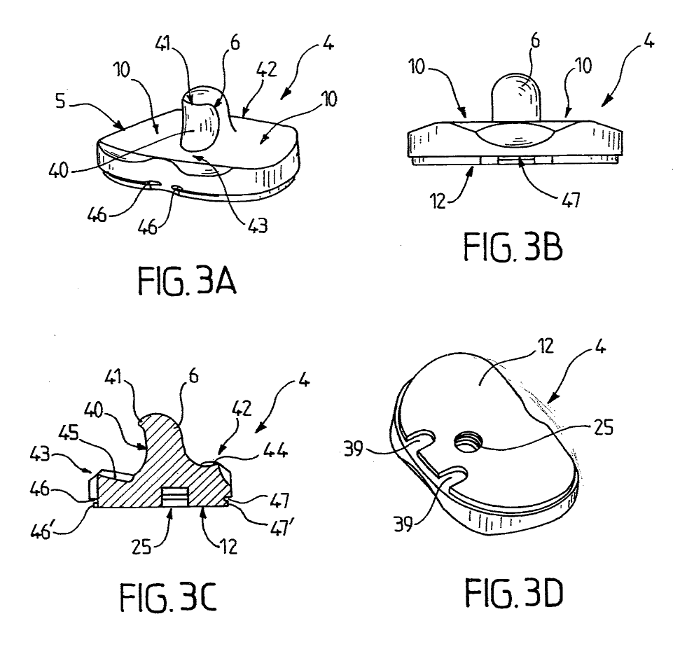

FIGS. 3A to 3D show an axonometric perspective view, a rear view, a lateral cross-sectional view and a bottom view, respectively, of the meniscal plate according to the embodiment of she invention more particularly described here.

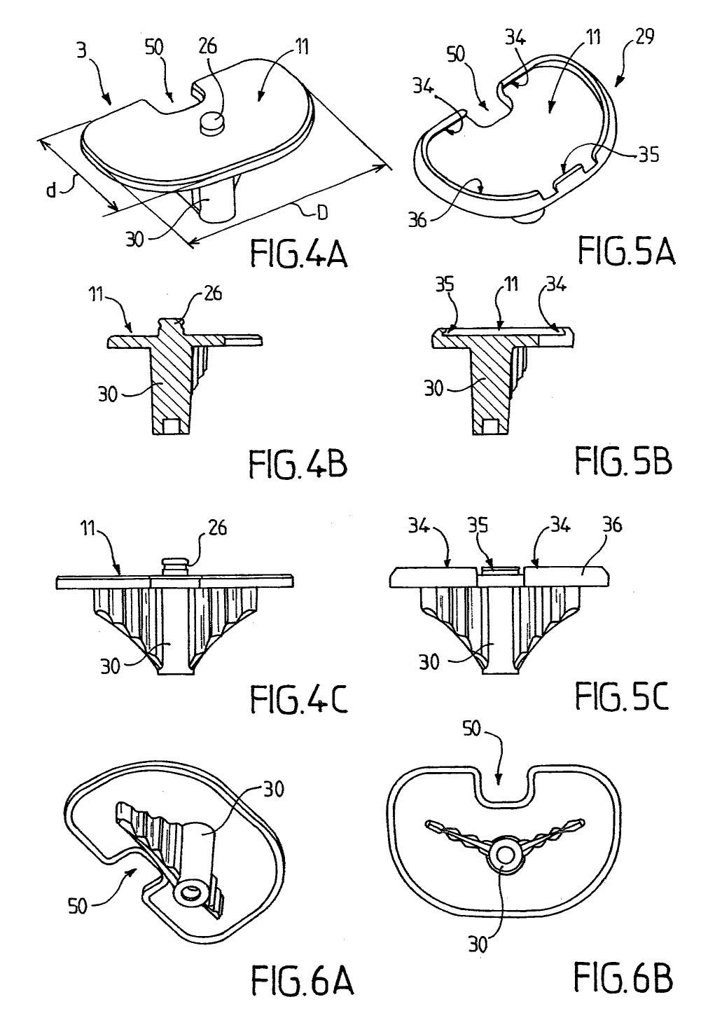

FIGS. 4A to 4C show an axonometric perspective view, a lateral cross-sectional view and a front view, respectively, of the tibial plateau according to the embodiment of FIG. 1.

FIGS. 5A to 5C show an axonometric perspective view, a lateral cross-sectional view and a front view, respectively, of the tibial plateau according to the embodiment of FIG. 2.

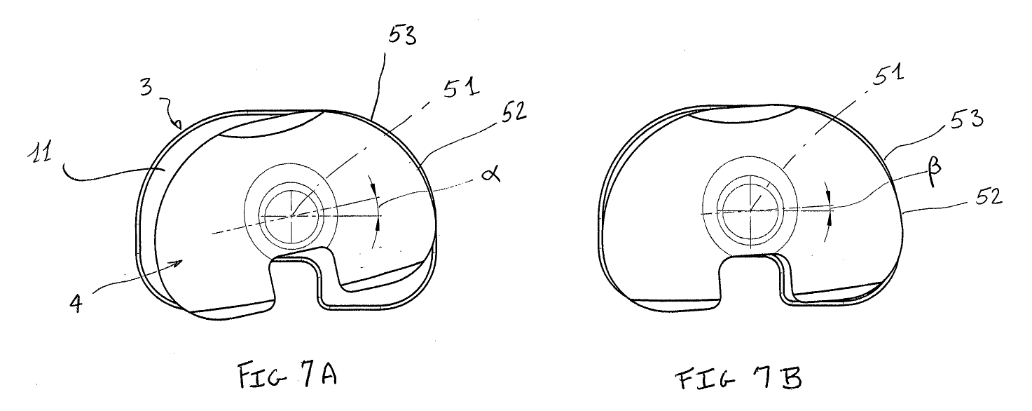

FIGS. 6A and 6B show a bottom view, in perspective, and a top view of an embodiment of the tibial plateau according to the invention.

FIGS. 7A and 7B each show, in a diagrammatic plan view, the relative positions of the joining face of the meniscal plate with respect to the joining face of the tibial plateau.

In the description below, the same reference numbers will be used to designate identical elements.

FIGS. 1A and 1B show a knee prosthesis 1 comprising a femoral component 2 connected to a tibial plateau 3 by an intermediate meniscal plate 4 provided with an upper face 5 comprising a pin 6. The femoral component 2 has a trochlear shield 7 provided with a slit 8 for guiding the pin, and an outer face 9 which cooperates, under gentle friction, with two guide areas 10 of complementary shape formed in the upper face 5 on each side of the pin.

The intermediate meniscal plate 4 and the tibial plateau 3 are connected to each other by way of two planar joining faces, namely a tibial joining face 11 and a meniscal joining face 12, the tibial joining face 11 being larger than the meniscal joining face 12 in its antero-posterior dimension d and medio-lateral dimension D.

The pin 6 is shaped like a Phrygian cap or the upper phalanx of a thumb, having an upper lip 13 forming a slight projection 14 designed to generate a retreating movement of the femoral component in the event of the latter shifting (arrow 15) by more than 1 mm with respect to the meniscal plate.

More precisely, the femoral component 2 comprises a first tongue-shaped part 16 ending at a point 16′, defining the concave surface of revolution of the outer sliding face 9, which has a variable radius of curvature between said first tongue-shape part and its lateral parts 17, symmetrical with respect to a central axis 18.

The femoral component terminates on the other side with end parts 19, opposite the pointed part of the trochlear surface, said parts 19 being curved inward.

In this embodiment, these two parts 19 are connected by way of a central joining beam 20, closing the guide slit 8 for the pin.

The slit 8 has, for example, a substantially rectangular shape with a curvature corresponding to that of the femoral component.

In the embodiment more particularly described here, the slit 8, with a width slightly greater than that of the pin 6, for example of 2 mm, has a length which, for example, is equal to two thirds of the deployed length of the surface of cooperation of the femoral component 2 wish the meniscal plate 4.

The meniscal plate 4 itself has a first part 21 anterior to the pin 6 and rising toward the outside, and a second part 22 posterior to the central pin of the meniscal plate, provided with a rounded reinforcement zone 23 and connecting the two postero-lateral zones 10 of complementary shape to the two lateral zones of the outer face 9 of the trochlear shield of the femoral component.

This reinforcement zone 23 can itself be provided with a recess 24 toward the outside, situated on its outer surface, permitting a better grip of the meniscal plate.

The meniscal plate 4 will be described in more detail below with reference to FIGS. 3A to 3D.

However, for the time being, it will be noted that this meniscal plate comprises, in combination, a cylindrical central orifice 25 (cf. FIG. 1B), in the form of a blind hole, capable of cooperating under gentle friction with a pivot pin 26 of the tibial plateau 3 (cf. FIG. 1A) of complementary cylindrical shape, and at lease one locking recess 27 (cf. FIG. 2B) capable of being engaged by an indexing finger 28 of the tibial plateau 29 (cf. FIG. 2B).

With reference to FIGS. 1A, 1B, 2A and 2B, the two embodiments of tibial plateau 3 or 29 usable with the prosthesis according to the invention will now be described.

Thus, in FIGS. 1A and 1B, a tibial plateau 3 has a central pivot pin 25 situated substantially at the center of the tibial joining face 11, which cooperates with the meniscal joining face 12.

The pin 26 is cylindrical and has, for example, an end forming a slight projection that engages with a snap fit in a complementary groove internal to the blind hole 25.

The tibial plateau 3 moreover has a bone-anchoring foot 30 of a form known per se, for example composed of a central plug 31 provided with two fluted lateral wings 32 forming a V shape, permitting good fixation and indexing in the bone once the latter has been resected by the surgeon in order to implant said tibial plateau therein.

FIGS. 2A and 2B show a prosthesis 33 according to the other embodiment of the invention more particularly described here, comprising the tibial plateau 29, which has a substantially similar shape but this time with two posterior indexing fingers 34 and one anterior indexing finger 35 (equivalent to the aforementioned finger 28). These will be seen more clearly by reference to FIG. 5B, which is described below.

In the embodiment in FIG. 2A, the anterior and posterior fingers are connected by the peripheral edge 36 of the plateau, namely a raised edge which, in the anterior part and the posterior part of the prosthesis, thus has clipping finger configurations.

However, the clipping can advantageously be effected about the entire periphery of the plateau, in which case the whole periphery 36 clips into an opposite recess 37 of the edge of the meniscal face.

Two tongue-shaped recesses 39 are also provided at an angle to the periphery of the meniscal joining face 12.

A tongue-shaped recess is, for example, of substantially oval or rectangular shape, with a length corresponding to a fifth or a seventh of the distance d of the meniscal face. When an instrument is slid into them, these recesses permit leverage and allow the meniscal plate to be disconnected from the tibial plateau when they have been fitted by clipping.

Advantageously, the peripheral edge 36 of the plateau is recessed slightly at 38 in the area of the “tongues” in order to facilitate the introduction of the tool forming the lever arm.

FIGS. 3A to 3D show in more detail the meniscal plate 4 that has been described with reference to FIG. 1.

This plate 4 thus has an upper face 5 provided with a pin 6, which has the shape of a Phrygian cap or the end of a thumb and which is provided with an inwardly curved part 40 ending with an upper lip 41 forming a slight projection. embodiment, to be without pin 10, especially But the plate 4 can of course, in another when the antero-posterior ligament is preserved by the surgeon.

The upper face 5 comprises, on each side of the anterior area 42 and posterior area 43 of the surface, raised parts 44 and 45, for example raised by 1 to 2 mm with respect to the adjacent surface. The face 5 also comprises, on each side of the pin, concave surfaces of development forming the guide parts 10.

The meniscal face 12 cooperating with the tibial face 11 has (cf. FIGS. 3C and 3D) the orifice 25 for joining to the pin 26 of a tibial plateau, for example permitting gentle clipping on account of the shape of the pin, as described hereinabove, and thereafter rotation, with gentle friction, of one relative to the other.

The face 12 also has (cf. FIG. 3C) recesses 46, 47 designed to cooperate with the opposite fingers 34, 35 of the tibial plateau, in the case where it is desired that this is blocked.

The recesses are formed, for example, by grooves with protuberances 46′, 47′ in the plane of the joining face 12.

The two embodiments of tibial plateaus described with reference to FIGS. 1 and 2 are shown in more detail in FIGS. 4A to 4C and 5A to 5C and, in bottom views, in FIGS. 6A and 6B.

In FIGS. 4A to 4C, the plateau 3 has the pin 26 permitting the rotation of the meniscal plate with respect to the plateau.

It is therefore a rotary plateau. The tibial joining face 11 has an antero-posterior dimension d and medio-lateral dimension D slightly greater than the meniscal face 12, so as not to protrude past the latter during the rotation, which can be done by a few degrees.

By virtue of the relative dimensions between meniscal joining face and tibial joining face, it will be possible to permit a rotation without external protrusion, even in the case of mixing sizes as necessitated by anatomical constraints.

For example, if one takes a femoral component of size 5, a meniscal plate of size 5 and a smaller tibial plateau, of size 4, the soft tissue parts of the knee still remain protected, even in the event of rotation by a few degrees.

This important feature in the embodiment of the invention more particularly described here will be explained in detail with reference to FIGS. 7A and 7B.

The embodiment in FIGS. 5A to 5C for its part describes a plateau for a fixed meniscal plate.

Here, the meniscal place is clipped rigidly into the base formed by the tibial plateau 29, which is here once again of a slightly greater size.

When, in the prior art, the sizes were organized to meet anatomical constraints, the differentiation was obtained especially with the femur.

According to the invention, with which it is possible to reduce by half the number of meniscal plates, the insert is always of a smaller size, allowing it to fit on a fixed base as well as a mobile base. It is therefore the fixed base or tibial plateau which, by virtue of its special configuration, adapts to the insert or meniscal plate.

Finally, the tibial joining face advantageously comprises an opened peripheral part 50 for passage of a finger under the meniscal joining face, allowing is to be disconnected from the plateau. This recess 50 is, for example, substantially rectangular, with a width of between 1/10th and ⅓rd, for example ¼th, of the dimension d, and with a length of between 1/10th and ⅓rd of D, for example ⅕th.

FIGS. 7A and 7B each show a plan view of she tibial plateau 3 from FIG. 1, onto which the meniscal plate 4 is clipped. The latter is able to pivot about the axis 51 of the pin 26, the joining faces 11 and 12 of the tibial plateau and of the meniscal plate, respectively, cooperating under gentle friction. The axis 51 of the pin 26 advantageously coincides with the axis of the pin 6.

When the meniscal plate pivots slightly about the axis 51 during the movements of the patient, it is observed that the outer peripheral edge 52 of the meniscal plane, having a cross section in the shape of a truncated ellipse, remains within the joining face 11, that is to say doss not protrude outward from the peripheral edge 53, of the tibial plateau for an angle α of the order of 12° when, normally, the components are of the same size (FIG. 7A), or for an angle β of the order of 4° when the tibial plateau is smaller than the meniscal plate.

The implantation of a prosthesis according to the invention by a surgeon is now described with reference to FIGS. 1A and 1B.

When the surgeon begins an operation, he first of all determines, from the physiology of the patient, what will best correspond to the bone configuration of the latter.

On this basis, he decides to fit either a fixed prosthesis or a mobile prosthesis.

He then chooses the tibial plateau corresponding to either the fixed or mobile solution.

This solution having been adopted, all he has to do is implant, according to the size of the patient, the femoral component that is standard regardless of whether the fixed type or mobile type has been chosen. Finally, it only remains for him to determine the size of the meniscal plate according to the tibial plateau and the patient's configuration.

Whereas in the prior art he had to choose, for example, from among ninety-six different meniscal plates depending on the tibial plateau, he now only has to choose from among forty-eight meniscal plates, by virtue of the invention.

As has been indicated, the savings both in time and in costs, especially storage costs, are considerable.

It goes without saying, and it is also obvious from the above, that the present invention is not limited to the embodiments that have been more particularly described. On the contrary, it encompasses ail variants thereof, especially those in which the meniscal plates are made of plastic and those in which the surfaces of cooperation between the trochlear shield of the femoral component and the meniscal plate are of different configurations, and those in which the meniscal plate does not have a pin on its upper face, especially in the case of surgery where the cruciate ligament is not removed.

1. A knee prosthesis (1, 33) comprising a femoral component (2) connected to a tibial plateau (3, 29) by an intermediate meniscal plate (4) provided with an upper face (5), said femoral component (2) having a trochlear shield (7) provided with an outer face (9) cooperating under gentle friction with at least one guide area (10) of complementary shape formed in said upper face, characterized in that
the intermediate meniscal plate (4) and the tibial plateau (3, 29) are connected to each other by way of two planar joining faces, namely a tibial joining face (11) and a meniscal joining face (12), the tibial joining face (11) being larger than the meniscal joining face in its antero-posterior dimension d and medio-lateral dimension D,
in that the tibial plateau (3, 29) comprises a central pivot pin (26) or at least one clipping/indexing finger (34, 35, 36), said pin or said finger protruding with respect to the tibial joining face (11),
and in that the meniscal joining face (12) of the meniscal plate (4) comprises, in combination, a central blind hole (25) capable of cooperating under gentle friction about its entire periphery with the pivot pin (26) of the tibial plateau in one case, and a locking recess (46, 47) capable of being engaged by the indexing finger (34, 35) of the tibial plateau in the other case, depending on whether the tibial plateau has a pivot pin or an indexing finger.

2. The prosthesis as claimed in claim 1, characterized in that the planar joining surfaces of the meniscal plate and of the tibial plateau are in the shape of an ellipse or substantially in the shape of an ellipse, truncated on an edge parallel to the main axis of said ellipse.

3. The prosthesis as claimed in claim 1, characterized in that the outer periphery of the meniscal joining face is designed to be inscribed within the joining surface of the tibial plate for an angle α of rotation between the two faces of between −12° and +12° with respect to the medio-lateral axis of the meniscal part.

4. The prosthesis as claimed in claim 3, characterized in that the angle α is between −4° and +4°.

5. The prosthesis as claimed in claim 1, characterized in that the upper face of the meniscal plate is provided with a pin and with two guide areas formed in the upper face on each side of said pin (16), the trochlear shield of the femoral face having a slit (8) for guiding said pin.

6. The prosthesis as claimed in claim 1, characterized in that the tibial plateau (29) comprises at least one indexing finger (34, 35), designed to engage in the recess (46, 47) for locking the meniscal plate in a defined position with respect to the tibial plateau.

7. The prosthesis as claimed in claim 6, characterized in that the tibial plateau comprises two posterior indexing fingers (34) and one anterior indexing finger (35) able to cooperate with two posterior recesses (46) and one anterior recess (47) of the meniscal plate.

8. The prosthesis as claimed in claim 1, characterized in that the tibial joining face (11) has a peripheral shoulder (36) forming a clipping finger along at least part of the periphery of said plateau in which the meniscal face engages completely.

9. The prosthesis as claimed in claim 1, characterized in that the tibial plateau (3) comprises a central pivot pin (26), the meniscal plate being mounted so as to be movable in rotation with respect to the tibial plateau about said pin.

10. The prosthesis as claimed in claim 1, characterized in that the pin (6) of the upper face of the meniscal plate has the shape of a Phrygian cap or a thumb.

11. The prosthesis as claimed in claim 10, characterized in that the Phrygian cap has an upper lip (41) forming a slight projection designed to generate a retreating movement of the femoral component in the event of its shifting by more than 1 mm with respect to the meniscal plate.

12. The prosthesis as claimed in claim 1, characterized in that the meniscal joining face (12) has at least one recess (39) angled with respect to the periphery of said face and designed to permit the disconnection of the meniscal plate from the tibial plateau.

13. The prosthesis as claimed in claim 1, characterized in that the zone (43) posterior to the central pin of the meniscal plate has a reinforcing part (45) connecting the two postero-lateral zones of articular friction.

14. The prosthesis as claimed in claim 1, characterized in that the femoral component (2) and the tibial plateau (3, 29) are made of metal alloy, and in that the meniscal plate is made of polymer plastic.

15. Set of prostheses as claimed in claim 1, characterized in that it has at least one tibial plateau (29) for a fixed mixed meniscal plate (4) and at least one tibial plateau (3) for a meniscal plate, and a range of corresponding mixed meniscal plates (4) of several thicknesses.
